(19) United States
(12) Patent Application Publication
(10) Pub. No.: US 2020/0330031 A1
(43) Pub. Date: Oct. 22, 2020

(54) SYSTEMS AND METHODS FOR ANESTHESIA MANAGEMENT

(71) Applicant: Neurolndex Ltd., Yokneam (IL)

(72) Inventor: Goded SHAHAF, Haifa (IL)

(73) Assignee: Neurolndex Ltd., Yokneam (IL)

(21) Appl. No.: 16/769,616

(22) PCT Filed: Dec. 5, 2018
(86) PCT No.: PCT/IB2018/001632

§ 371 Date: Jun. 4, 2020

Related U.S. Application Data
(60) Provisional application No. 62/685,819, filed on Jun. 15, 2018.
(60) Provisional application No. 62/595,021, filed on Dec. 5, 2017.

(51) Int. Cl. A61B 5/00; A61B 5/0482; A61B 5/0484; A61B 5/04

(52) U.S. Cl. CPC A61B 5/4821; A61B 5/0482; A61B 5/0484; A61B 5/742; A61B 5/7275; A61B 5/04017; A61B 5/6814

(57) ABSTRACT

Systems and methods are disclosed for management of anesthetized patients. The systems and methods can enable determination of whether the patient risks an anesthetic complication, an intraoperative stroke, or awareness during anesthesia. The disclosed systems and methods can involve receiving a signal from EEG electrodes on the head of a patient during presentation of a stimuli to the patient. This signal can be used to generate segment posteriorization index values and/or synchronization values. Based on synchronization values anesthesia depth of the patient can be reduced or a surgical intervention performed. Based on segment posteriorization index values anesthesia depth of the patient can be increased.

### TECHNICAL FIELD

The disclosed embodiments concern systems, methods, and devices for assessing and managing the cognitive effects of anesthesia and sedation on a patient.

### SUMMARY

The disclosed embodiments include systems and methods for anesthesia management using an electroencephalographic (EEG) monitoring device. More specifically, the disclosed embodiments concern systems, methods, and devices for real-time determination of systemic and/or focal brain dysfunction using electroencephalography. The disclosed embodiments can also be used to monitor a depth of anesthesia of the patient and/or identify a potential stoke, intraoperative stroke, concussion, or traumatic brain injury.

The American Society of Anesthesiologists (ASA) has acknowledged that cognitive changes and delirium after surgery are major health challenges that needs to be addressed. An estimated of 12%-50% of surgical patients, depending on variety of risk factors including increased age, experience Post-Operative Delirium (POD). The reported incidence of Post-Operative Cognitive Dysfunction (POCD) also varies. The incidence of POCD after cardiac surgery for example, is reported to be 30-80% while in major non-cardiac surgeries 30-40% of adults of all ages were diagnosed with POCD. More than one-third of inpatient surgeries in the U.S. are currently performed on patients 65 years or older and that number is expected to rise as the proportion of the U.S. population over age 65 continues to increase dramatically in the coming decades. The direct costs to the hospital system of POCD is in the range of $18-20 billion annually. Estimates suggest that caring for patients with POD and POCD costs more than $150 billion a year.

Excessive anesthesia is associated with increased risk of POD and POCD, but insufficient anesthesia is associated with risk of patient recall or awareness during an operation. The Bispectral Index (BIS) is used to measure the depth of anesthesia in anesthetized patients. This index depends on the electroencephalogram and electromyogram of the patient. Devices for calculating the Bispectral Index (BIS devices) have been widely adopted in Anesthesiology. In general, EEG-based depth of anesthesia monitors are recommended during general anesthesia for patients with a high risk of unintended awareness or a high risk of excessively deep anesthesia.

But BIS does not necessarily provide an accurate measure of the depth of anesthesia. For example, BIS values can be drug-dependent. Patients administered drugs causing neuromuscular block can have anomalous BIS values. Such patients can appear deeply anesthetized according to their BIS values when they are actually only lightly anesthetized. As a result, such a patient may be inadequately anesthetized during an operation, causing her to remember the operation, or experience anxiety, awareness, or pain during the operation.

The disclosed embodiments include a first method for treating an anesthetized patient. The first method can include a sequence of operations. The operations include determining whether the patient risks awareness during anesthesia. This determination can be accomplished at least in part by receiving at least one signal from EEG electrodes on the head of the anesthetized patient during presentation of a stimuli to the patient and generating segment posteriorization index values to determine whether the patient risks awareness during anesthesia. The EEG electrodes can include an anterior electrode and a posterior electrode. Generating segment posteriorization index values can include generating filtered EEG signal epochs using the at least one signal; generating epoch values using the filtered EEG signal epochs; and generating the segment posteriorization index values using the epoch values. If segment posteriorization index values satisfy a risk criterion for awareness during anesthesia then a depth of anesthesia of the anesthetized patient can be increased. If segment posteriorization index values do not satisfy the risk criterion for the anesthetic complication then a depth of anesthesia of the anesthetized patient can be maintained.

Increasing the depth of anesthesia of the anesthetized patient can include administering an anesthetic, increasing a rate of administration of an anesthetic, or administering an anesthetic agonist. Generating the filtered EEG signal epochs can include filtering the at least one signal using a filter having at least one of a lower cutoff frequency of 5-9 hertz and at higher cutoff frequency of 11-15 Hz. Generating the epoch values using the filtered EEG signal epochs can include identifying valid epochs and generating epoch values using valid epochs. Identifying valid epochs can further include identifying valid segments. Determining whether the patient risks awareness during anesthesia further can further include generating a global posteriorization index value based on the segment posterior index values. Satisfaction of the risk criterion can depends on the global posteriorization index value. Generating epoch values using the filtered EEG signal epochs can include identifying invalid epochs and/or invalid segments. Epochs can be invalid when a filtered EEG signal does not satisfy a relative amplitude criteria during the epoch. Segments can be invalid when a percentage of the epochs comprise the segments are invalid. The epoch values can include anterior values based on EEG signals for the at least one anterior electrode and posterior values based on EEG signals for the at least one posterior electrode.

The disclosed embodiments include a first diagnostic device. The second diagnostic device can include at least one processor; and at least one computer readable medium. The computer readable medium can store instructions that, when executed by the at least one processor, cause the diagnostic device to perform operations. The operations can include receiving at least one signal from EEG electrodes on the head of a patient during presentation of a stimuli to the patient. The EEG electrodes can include at least one anterior electrode and at least one posterior electrode. The operations can further include generating filtered EEG signal epochs using the at least one signal; generating epoch values using the filtered EEG signal epochs; generating segment posteriorization index values using the epoch values; and displaying an indication based on the segment posteriorization index values.

Generating the filtered EEG signal epochs can include filtering the at least one signal using a filter having at least one of a lower cutoff frequency of 5-9 hertz and at higher cutoff frequency of 11-15 Hz. Generating the epoch values using the filtered EEG signal epochs can include identifying valid epochs and generating epoch values using valid epochs. The operations can further include generating a global posteriorization index value based on the segment posterior index values. The indication can be based on this global posteriorization index value. Generating epoch values using the filtered EEG signal epochs can include identifying invalid epochs and/or invalid segments. Epochs can be invalid when a filtered EEG signal does not satisfy a relative amplitude criteria during the epoch. Segments can be invalid when a percentage of the epochs comprising the segments are invalid. The epochs can be between 500 ms and 3 seconds in duration. Segments can include between 5 and 20 consecutive epochs. The epoch values can include anterior values based on EEG signals for the at least one anterior electrode and posterior values based on EEG signals for the at least one posterior electrode. Generating segment posteriorization index values using the epoch values can include determining that a count of valid epochs in a segment satisfy a relative epoch value criterion. Valid epochs with posterior values greater than anterior values can satisfy the relative epoch value criterion.

The disclosed embodiments include a second method for treating an anesthetized patient. The second method can include a sequence of operations. The operations can include determining whether the patient risks an anesthetic complication. This determination can be performed at least in part by receiving at least one signal from a pair of EEG electrodes on the head of the patient during presentation of a stimuli to the patient and generating a synchronization value to determine whether the patient risks awareness during anesthesia. The pair of electrodes can include a left hemisphere electrode and a right hemisphere electrode. Generation can include generating filtered EEG signal epochs using the at least one signal; generating epoch values using the filtered EEG signal epochs; calculate a synchronization value using the epoch values; and displaying an indication based on the synchronization value. If the synchronization values satisfy a risk criterion for the anesthetic complication then a depth of anesthesia of the anesthetized patient can be reduced or a surgical intervention can be performed. If the synchronization values do not satisfy the risk criterion for the anesthetic complication a depth of anesthesia of the anesthetized patient can be maintained.

The anesthetic complication can include post-operative delirium, post-operative cognitive deterioration, relative hypotension, relative hypoxia, or relative hypoglycemia. Reducing the depth of anesthesia of the anesthetized patient can include delaying administration of an anesthetic dose, reducing a rate of administration of an anesthetic, or administering a reversal agent. The surgical intervention can include a thombectomy. The operations can further include providing an indication of an intraoperative stroke if the synchronization values satisfy an intraoperative stroke risk criterion.

Generating the filtered EEG signal epochs can include filtering the at least one signal using a filter having at least one of a lower frequency cutoff of 0.5-2 Hz and an upper frequency cutoff of 3-5 Hz. The epoch values can include statistical measures of filtered EEG signals for the first electrode and the second electrode. The epoch values can include a set of epoch values for the left hemisphere electrode and a set of epoch values for the right hemisphere electrode. The synchronization value can include a Pearson's correlation coefficient or a Spearman correlation coefficient calculated between the set of epoch values for the left hemisphere electrode and the set of epoch values for the right hemisphere electrode. Calculating the synchronization value can include identifying a set of consecutive valid epochs having a predetermined minimum size greater than 5 consecutive epochs and/or a predetermined maximum size less than 60 consecutive epochs. Valid epochs can satisfy a relative amplitude criteria.

The disclosed embodiments include a second diagnostic device. The second device can include at least one processor and at least one computer readable medium. The computer readable medium can store instructions that, when executed by the at least one processor, cause the diagnostic device to perform operations. The operations can include receiving at least one signal from a pair of EEG electrodes on the head of a patient during presentation of a stimuli to the patient. The pair can include a left hemisphere electrode and a right hemisphere electrode. The operations can further include generating filtered EEG signal epochs using the at least one signal; generating epoch values using the filtered EEG signal epochs; calculate a synchronization value using the epoch values; and displaying an indication based on the synchronization value.

The indication can concern whether the patient is experiencing or experienced a concussion or a stroke. The indication can concerns whether the patient has focal brain damage. Generating the filtered EEG signal epochs can include filtering the at least one signal using a filter having at least one of a lower frequency cutoff of 0.5-2 Hz and an upper frequency cutoff of 3-5 Hz. The epoch values can include statistical measures of filtered EEG signals for the first electrode and the second electrode. The epoch values can include a set of epoch values for the left hemisphere electrode and a set of epoch values for the right hemisphere electrode. The synchronization value can include a Pearson's correlation coefficient or a Spearman correlation coefficient calculated between the set of epoch values for the left hemisphere electrode and the set of epoch values for the right hemisphere electrode. Calculating the synchronization value can include identifying a set of consecutive valid epochs having a predetermined minimum size greater than 5 consecutive epochs and/or a predetermined maximum size less than 60 consecutive epochs. The left hemisphere electrode and the right hemisphere electrode can be symmetrically placed on the head of the patient. The left hemisphere electrode and the right hemisphere electrode can be frontal electrodes. The stimuli can be an auditory oddball task.

It is to be understood that both the foregoing general description and the following detailed description are exemplary and explanatory only and are not restrictive of the disclosed embodiments, as claimed.

### BRIEF DESCRIPTION OF THE DRAWINGS

The drawings are not necessarily to scale or exhaustive. Instead, emphasis is generally placed upon illustrating the principles of the embodiments described herein. The accompanying drawings, which are incorporated in and constitute a part of this specification, illustrate several embodiments consistent with the disclosure and, together with the description, serve to explain the principles of the disclosure. In the drawings:

FIGS. 1A-1E depicts components of various exemplary event-related potentials.

FIG. 2A depicts an exemplary comparison of BIS values in three clinical states.

FIG. 2B depicts an exemplary comparison of indices generated according envisioned systems and methods for four clinical states.

FIG. 3 depicts an exemplary comparison of the dependence on normalized EMG of BIS and the envisioned index.

FIG. 4 illustrates the diagnostic implications of a localized dysfunction index and a spread dysfunction index.

FIG. 5 depicts an exemplary analysis of EEG signals.

FIG. 6 depicts an exemplary device for acquiring EEG signals.

FIG. 7 depicts an exemplary method for adjusting the placement of the exemplary device of FIG. 6 on a patient.

FIG. 8 depicts method for detecting systemic brain dysfunction.

FIG. 9 depicts method for detecting focal brain dysfunction.

FIGS. 10A-10F depict the results of a study demonstrating that synchronization index values can identify anesthetized patients at risk of POCD.

FIGS. 11 to 12D depict the results of a study demonstrating that posteriorization index values can identify anesthetized patients at risk of awareness during anesthesia.

FIG. 13 depicts an exemplary criterion for determining appropriate sedative dosing to avoid both delirium and recall.

FIG. 14 depicts the results of a study demonstrating that synchronization index values can identify anesthetized patients potentially experiencing an intraoperative stroke.

FIG. 15 depicts a method for treating an anesthetized patient who risks an anesthetic complication.

FIG. 16 depicts a method for treating an anesthetized patient who risks awareness during anesthesia.

FIG. 17 depicts the results of a study demonstrating that that synchronization index values can identify patients with concussions.

### DETAILED DESCRIPTION

Reference will now be made in detail to exemplary embodiments, discussed with regards to the accompanying drawings. In some instances, the same reference numbers will be used throughout the drawings and the following description to refer to the same or like parts. Unless otherwise defined, technical and/or scientific terms have the meaning commonly understood by one of ordinary skill in the art. The disclosed embodiments are described in sufficient detail to enable those skilled in the art to practice the disclosed embodiments. It is to be understood that other embodiments may be utilized and that changes may be made without departing from the scope of the disclosed embodiments. Thus the materials, methods, and examples are illustrative only and are not intended to be necessarily limiting.

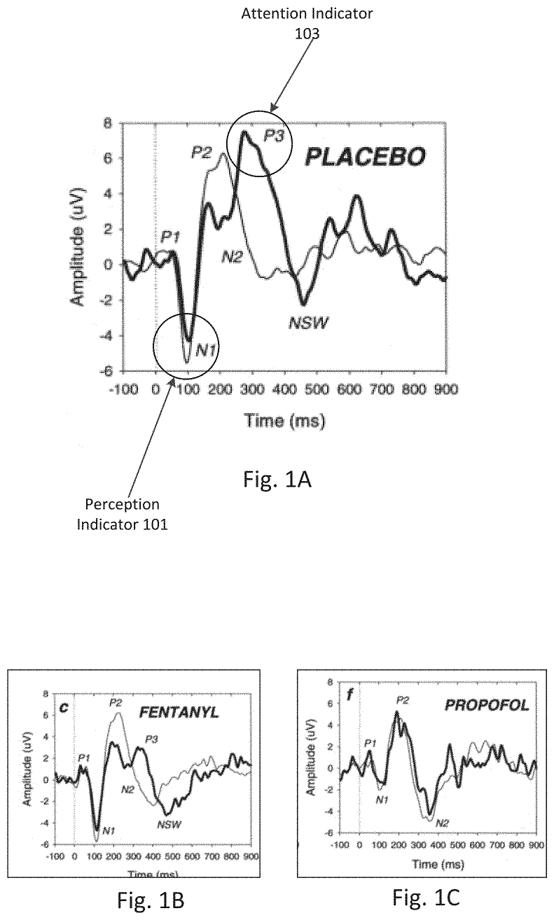

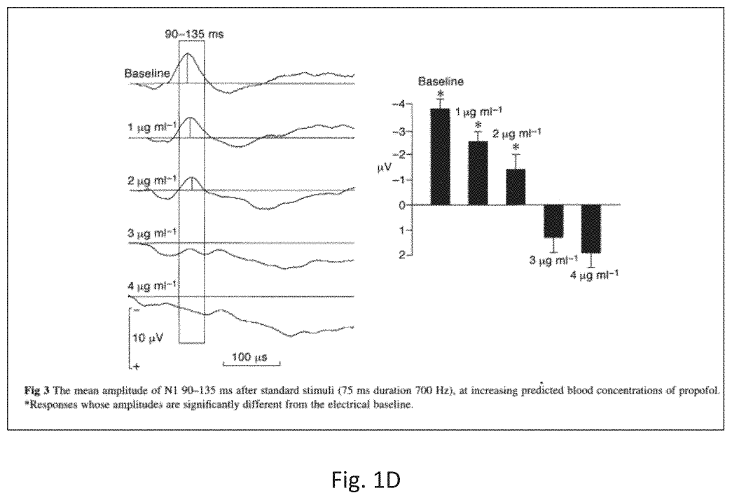

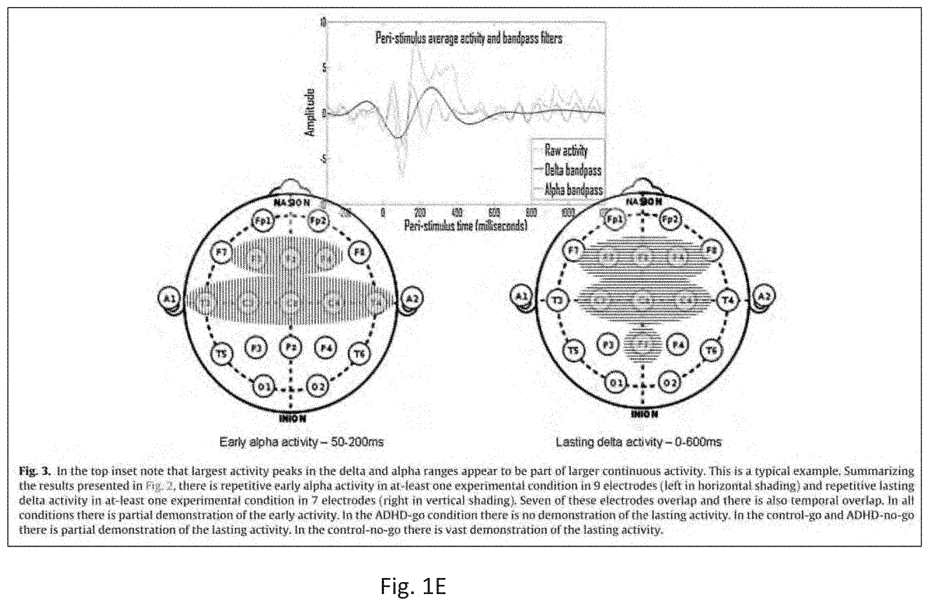

Certain disclosed embodiments may use event-related potentials (ERPs) to improve upon existing methods for detection of patient brain dysfunction and measurement of patient depth of anesthesia. As known in the art, an ERP is a stereotyped electrophysiological response to a stimulus. ERPs can be evoked, for example, using visual, auditory, or tactile stimuli. FIG. 1A depicts components of an exemplary ERP, consistent with disclosed embodiments. Depicted components of the exemplary ERP include several positive-going peaks (P1, P2, and P3) and several negative going peaks or waves (N1, N2, and NSW). These peaks and waves reflect the coordinated action of large numbers of neurons and are localized to certain neuroanatomic structures. These neuroanatomic structures are also associated with certain metal processes, such as memory, attention, and perception. Drugs affect activity in these neuroanatomic structures, leading to measurable effects on ERPs. Therefore, the effect of a drug on ERPs can be associated with an effect of the drug on activity in a neuroanatomic structure. The effect on the neuroanatomic structure can result in an effect on a clinical parameter of the patient, such as memory, attention, or perception. Thus, ERPs provide a mechanism for estimating the effect of a specific drug on a specific neurobiological process.

As shown in FIGS. 1A to 1D, the N1 and P3 peaks of an auditory ERP can be associated with a degree of anesthesia and sedation in a patient. The N1 peak is associated with perception (perception indicator 101), the gating of stimulus into the central nervous system. The P3 peak is associated with attention (attention indicator 103) the processing of stimulus. A patient sedated with fentanyl exhibited a reduction in the P3 peak (attention), without exhibiting a reduction in the N1 peak (perception). In contrast, a patient anesthetized with Propofol exhibits both a reduction in the N1 peak (perception) and the absence of a P3 peak (attention). These changes in ERP are dosage dependent. For example, as shown in FIG. 1D, the N1 peak diminishes with increasing blood concentration of Propofol. Furthermore, the components of auditory ERP exhibit a spatial dependence. EEG measurements using posterior electrodes can exhibit a more pronounced N1 peak during ERPs, while EEG measurements using anterior electrodes can exhibit a more pronounced P3 peak during ERPs. Filtered EEG data, like raw EEG data, can exhibit changes during an auditory ERP indicative of perception and attention. For example, as shown in FIG. 1E, a patient can exhibit repetitive early alpha activity and repetitive lasting delta activity during an auditory ERP. In some aspects, peri-stimulus alpha or delta activity of different electrodes (e.g., posterior and anterior electrodes), evoked using an auditory oddball task, can be used to evaluate attention and perception. In this manner, an ERP (such as an auditory ERP) can provide a real-time measure of the effect of administered drugs on the central nervous system functioning of a patient.

As shown, sedative-hypnotic drugs can produce profound changes in ERP waveforms (such as auditory ERP waveforms). Increasing anesthesia can decrease attention, which can be quantified by measuring P3 using anterior electrodes. Increasing anesthesia further can decrease perception, which can be quantified by measuring N1 using posterior electrodes. In contrast, BIS devices rely on anterior electrodes, limiting detection of N1, and are based on EEG/EMG, potentially confounding EEG and EMG measurements.

The disclosed systems and methods can monitor depth of anesthesia using measurements of attention and perception derived from ERP waveforms. These measurements can be derived by identifying specific ERP waveform patterns. These patterns can be identified using templates. In some embodiments, the disclosed systems and methods can use multiple electrodes. For example, the disclosed systems and methods can parietal electrodes (e.g., the P3 and P4 electrode locations in the 10-20 electrode setup). Additionally or alternatively, the disclosed systems and methods can use frontal electrodes (e.g., the F3 and F4 electrode locations in the 10-20 electrode setup).

In some embodiments, the disclosed systems and methods can calculate a systemic brain dysfunction index representative of a clinical state, such as depth of anesthesia. This index can be calculated using ERP patterns. In some aspects, this index can be calculated over a short sampling period, enabling temporally specific measurements of depth of anesthesia. This index can be independent of EMG. In some aspects, this index can be capable of differentiating between clinical states (e.g., light sedation with recall and light sedation without recall). Thus this systemic brain dysfunction index can assist clinicians in determining whether current anesthesia levels are sufficient to avoid recall during sedation. In this manner, the envisioned systemic brain dysfunction index can prevent unnecessarily high anesthesia dosages that could put a patient at risk for POCD or POD.

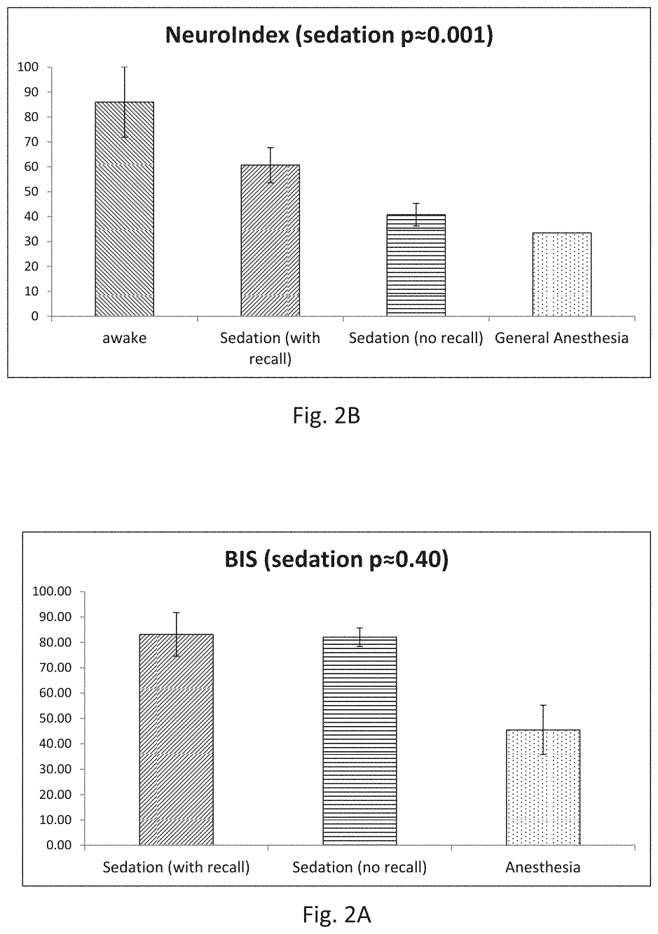

FIGS. 2A and 2B depict the improvement provided by the envisioned index over BIS indices. Three groups of patients were evaluated: patients in general anesthesia, light sedation, and deep sedation. Patients were monitored intraoperatively using standard ASA monitors including a pulse oximeter, electrocardiography, noninvasive blood pressure device, and a temperature monitor; a BIS monitor; and an EEG monitor (an EMOTIV EPOC) configured according to the disclosed systems and methods. In this study, the BIS monitor used anterior (frontal) electrodes, while the EEG monitor used both anterior (frontal) and posterior (parietal) electrodes. In the post-anesthesia care unit, patients completed a Brice Questionnaire to assess intraoperative awareness. Patients were categorized based on assessment of recall. FIG. 2A depicts an exemplary comparison of BIS in three clinical states. As shown, there was no significant difference in BIS between patients having sedation with recall (light sedation) and having sedation without recall (deep sedation). FIG. 2B depicts an exemplary comparison of indices generated according envisioned systems and methods for four clinical states. In contrast with BIS, the envisioned index calculated by the EEG monitor configured according to the disclosed systems and methods could distinguish between sedation with recall and sedation without recall. As would be appreciated by one of skill in the art, in certain circumstances there may be no clinical relevant difference between deep sedation and general anesthesia.

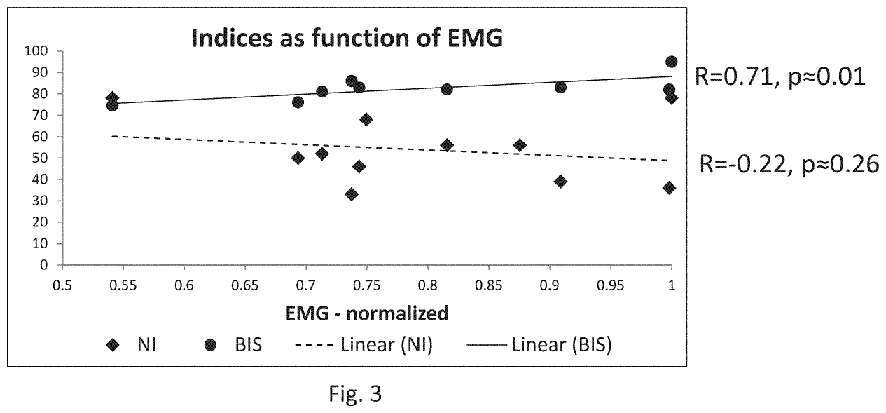

FIG. 3 depicts an exemplary comparison of the dependence on normalized EMG of BIS and the envisioned index. As shown, BIS is an increasing function of normalized EMG (p-value approximately 0.01). In contrast, the envisioned index appears independent of normalized EMG (p-value approximately 0.26). As would be appreciated by one of skill in the art, the dependence of BIS on normalized EEG can result in under-anesthetizing patients administered drugs causing neuromuscular block.

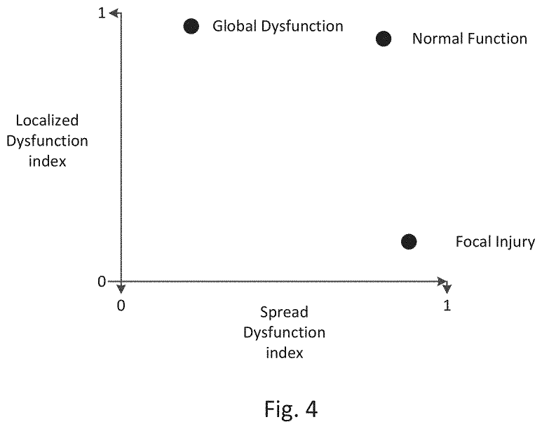

FIG. 4 illustrates the diagnostic implications of a localized dysfunction index and a spread dysfunction index. As shown in FIG. 4, neural signals can be used to determine a Localized Dysfunction Index (LDI) or a Spread Dysfunction Index (SDI).

The SDI can indicate a global level of brain function. The SDI of an anesthetized patient can be affected by level of anesthesia, and can be a predictor of post-operative delirium. Low values of SDI are indicative of global dysfunction. The SDI value can depend on posterior perceptual activity. This posterior perceptual activity can be normalized based on frontal activity. The posterior and frontal activity can be determined using electroencephalography. For example, the posterior and frontal activity can be derived from the alpha content of an electroencephalogram of the patient (e.g., the content of the electroencephalogram within a frequency range of approximately 7-13 Hz). The posterior perceptual activity and frontal activity can be determined bilaterally (e.g., using signals from two frontal electrodes and two posterior electrodes). The determining of the SDI value can occur during testing, upon cessation of testing, or after testing. The determination can occur using a recorded electroencephalogram.

The LDI can indicate a focal brain injury. The LDI of an anesthetized patient can be affected by dysfunction of hippocampal networks or stroke. The LDI can be a predictor of post-operative cognitive dysfunction. Low values of the LDI are indicative of focal injury. The LDI can depend on a degree of synchronization between the right and left hemispheres of frontal activity levels. For the purpose of the LDI, these activity levels can be derived from the delta content of an electroencephalogram of the patient (e.g., the content of the electroencephalogram less than approximately 4 Hz). A change in an LDI value for a patient can be compared to previously determined changes in LDI value for other patients. The diagnostic significance of a change in LDI value can depend on context—patients with strokes can exhibit a greater change in LDI values than patients with concussions. Thus the previously determined changes in LDI value for other patients can be used to gauge the diagnostic significance of a measured LDI change in the current patient.

According to the envisioned systems and methods, differential diagnosis of global dysfunction versus focal injury can involve calculating the LDI and SDI. In some implementations, this calculation can include normalizing the LDI and SDI to the clinical population. The differential diagnosis can depend on which of the LDI and SDI is lower. For example, in an operating room context, an SDI lower than LDI can be indicative of overuse of anesthetics (potentially leading to post-operative delirium), while an LDI lower than SDI can be indicative of hypoxia, hypotension, hypoglycemia, emboli, or focal injuries (potentially leading to chronic post-operative cognitive deterioration). As another example, in an emergency room context, an SDI lower than LDI can be indicative of a drug overdose or systemic disease, while an LDI lower than SDI can be indicative of a stroke, encephalitis, or a seizure. As a further example, when diagnosing suspected head trauma, an SDI lower than LDI can be indicative of severe pain or anxiety, while an LDI lower than SDI can be indicative of a concussion or cerebral hemorrhage. In general, normal brain function can be indicated by an SDI greater than a threshold value together with an LDI greater than a threshold value.

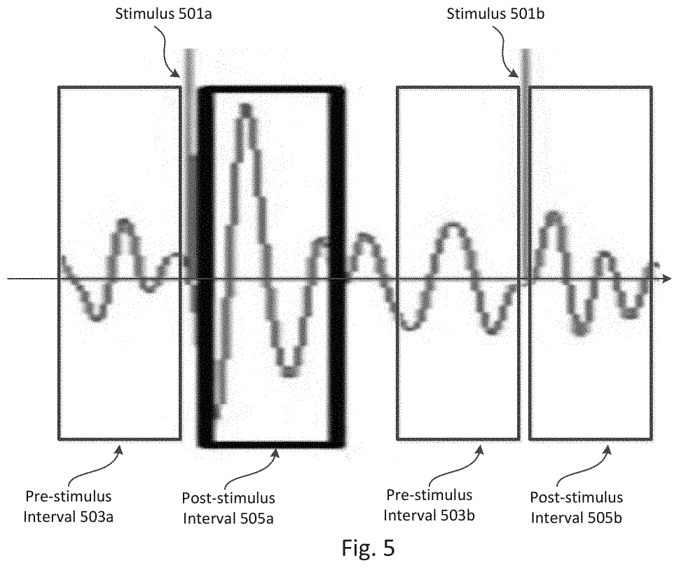

FIG. 5 depicts an exemplary SDI analysis of EEG signals. As shown in FIG. 5, a train of stimuli can be provided to a patient (e.g., stimulus 501a and stimulus 501b). In some embodiments, the stimuli can be auditory stimuli. For example, the train of stimuli can constitute an auditory oddball task, as would be familiar to one of skill in the art. In such a train of stimuli, approximately 70% to 90% of the stimuli in the train can be at a first pitch (e.g., 10000 Hz) and the remaining stimuli in the train can be at a second pitch (e.g., 2000 Hz). In some embodiments, the SDI analysis can be limited to the stimuli provided at a single pitch (e.g., the second pitch). In various embodiments, the SDI analysis can include stimuli provide at both the first and the second pitches. In some embodiments, a train of stimuli can include 2-20, or 5-15, stimuli (or such a number of oddball stimuli in a train of stimuli including additional non-oddball stimuli). Alternatively, or additionally, visual or tactile oddball stimuli may be presented to the patient.

In some embodiments, the SDI analysis can be limited to a peri-stimulus interval for each stimulus in the train of stimuli. This peri-stimulus interval can include a pre-stimulus interval (e.g., pre-stimulus interval 503a, pre-stimulus interval 503b) and a post-stimulus interval (e.g., post-stimulus interval 505a, post-stimulus interval 505b). The pre-stimulus interval can range in duration from 100 ms to 500 ms. The pre-stimulus interval can end either when the stimulus occurs or before the stimulus occurs. For example, the pre-stimulus interval can end 0-100 ms before the stimulus occurs. The post-stimulus interval can range in duration from 100 ms to 500 ms. The post-stimulus interval can begin either when the stimulus occurs or after the stimulus occurs. For example, the post-stimulus interval can begin 0-100 ms after the stimulus occurs.

In some embodiments, the SDI analysis can include calculating at least one activity level for the peri-stimulus interval. As an additional example, the SDI analysis can include calculating a pre-stimulus activity level and a post-stimulus activity level. Activity levels can be calculated from the power of an EEG signal, or from the power of the EEG signal in a particular frequency band, such as the power in the alpha frequency band (e.g., approximately 7-13 Hz). The pre-stimulus activity level can be calculated from the power of the EEG signal during the pre-stimulus interval. The post-stimulus activity level can be calculated from the power of the EEG signal during the post-stimulus interval. In some embodiments, the power of the signal can depend on either the integral or the average of a function of the EEG signal. This function can include the absolute value of the EEG signal. Alternatively or additionally, this function can include the exponentiation of the EEG signal (e.g., the square of the EEG signal). For example, the pre-stimulus activity level can depend on the integral of the absolute value of the EEG signal during the pre-stimulus interval. Similarly, the post-stimulus activity level can depend on the integral of the absolute value of the EEG signal during the post-stimulus interval.

In some embodiments, the SDI analysis can include calculating a change in activity levels between the pre-stimulus activity level and the post-stimulus activity level. For example, the SDI analysis can include calculating the difference between the pre-stimulus activity level and the post-stimulus activity level, or the absolute value of the difference between the pre-stimulus activity level and the post-stimulus activity level.

In some embodiments, the SDI analysis can include thresholding the change in activity levels between the pre-stimulus activity level and the post-stimulus activity level. For example, the SDI analysis can include identifying the peri-stimulus intervals for which the magnitude of this change in activity levels exceeds a value. As an additional non-limiting example, given a first set of stimuli in a train (or a first set of oddball stimuli in a train including additional non-oddball stimuli), the SDI analysis can include determining a second set of corresponding peri-stimulus intervals for which the magnitude of the change in activity levels exceeds a value.

In some embodiments, the SDI analysis can exclude peri-stimulus intervals that fail to satisfy certain criteria. For example, the SDI analysis can exclude “noisy” peri-stimulus intervals. Excluding such “noisy” peri-stimulus intervals from the SDI analysis can reduce the effects of changes in electrode conductance. In some aspects, “noisy” peri-stimulus intervals include peri-stimulus intervals for which an activity level exceeds a maximum threshold or fails to exceed a minimum threshold can be excluded from the analysis. As an additional example, the SDI analysis can exclude “unaffected” peri-stimulus intervals. In some aspects, “unaffected” peri-stimulus intervals include peri-stimulus intervals for which a change in activity level between the pre-stimulus activity level and the post-stimulus activity level exceeds a threshold. In some embodiments, “noisy” peri-stimulus intervals can be excluded before identification of “unaffected” peri-stimulus intervals. In various embodiments, the SDI analysis can first exclude “noisy” peri-stimulus intervals and then exclude, from among the remaining peri-stimulus intervals, the “unaffected” peri-stimulus intervals. In various embodiments, the SDI analysis can first exclude “unaffected” peri-stimulus intervals and then exclude, from among the remaining peri-stimulus intervals, the “noisy” peri-stimulus intervals.

In some embodiments, the SDI analysis can include determining SDI based on the non-excluded peri-stimulus intervals. In some aspects, SDI can depend on the ratio of a posterior activity level over an anterior activity level. The posterior activity level can be the maximum of the posterior activity levels measured for multiple electrodes (e.g., the maximum of the activity levels measured for electrodes on P3 and P4). The anterior activity level can be the maximum of the anterior activity levels measured for multiple electrodes (e.g., the maximum of the activity levels measured for electrodes on F3 and F4).

In some embodiments, the SDI analysis can include comparing a change in SDI for the patient with changes in SDI calculated for other patients. In some aspects, the other patients can include a relevant patient population. For example, when evaluating depth of anesthesia, the change in SDI for the patient during anesthesia can be compared to the change in SDI for other patients during anesthesia. Similarly, when monitoring a patient at risk for delirium, changes in SDI for the patient can be compared to changes in SDI for other patients during such monitoring. The SDI analysis can include determining whether the patient exhibits a condition based on the comparison of the change in SDI for the patient with the changes in SDI calculated for other patients.

In some embodiments, an LDI analysis can be performed, similar to the SDI analysis described above. As described above with regard to FIG. 5, a train of stimuli can be provided to a patient (e.g., stimulus 501a and stimulus 501b). This train of stimuli can have the same characteristics as described above with regard to the SDI analysis. In some embodiments, the LDI analysis can include calculating at least one activity level for the peri-stimulus interval, similar to the SDI analysis described above. The activity level for the LDI analysis can be calculated from the power of an EEG signal, or from the power of the EEG signal in a particular frequency band, such as the power in the delta frequency band (e.g., less than approximately 4 Hz). Activity levels can be calculated for multiple anterior electrodes (e.g., electrodes F3 and F4) and for multiple posterior electrodes (e.g., electrodes P3 and P4).

In some embodiments, the LDI analysis can include only peri-stimulus intervals that satisfy certain criteria. For example, the LDI analysis can include only peri-stimulus intervals in which an activity level for the anterior electrodes (e.g., a maximum of the activity levels for the anterior electrodes or a mean of the activity levels for the anterior electrodes) is greater than an activity level for the posterior electrode (e.g., a maximum of the activity levels for the posterior electrodes or a mean of the activity levels for the posterior electrodes). At times there is strong posterior activity under reduced consciousness (e.g., sleep spindles). Such activity can manifest asymmetrically in anterior electrodes. Excluding peri-stimulus intervals in which an activity level for the anterior electrodes is less than an activity level for the posterior electrode can reduce the impact of this posterior activity on the calculation of LDI.

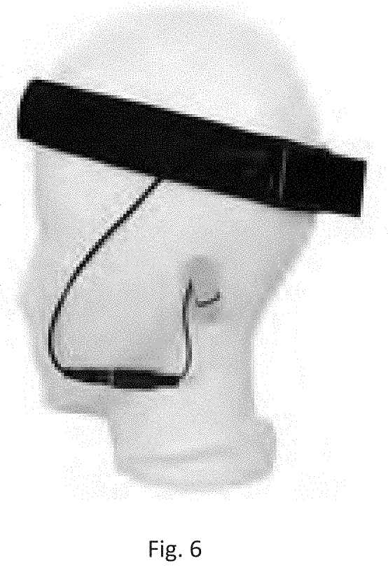

FIG. 6 depicts an exemplary device for acquiring EEG signals. In some embodiments, this device can include a flexible component that conforms to the head of a patient (e.g., a headband). The flexible component may be configured for placement around the head of the patient as shown in FIG. 6, with an anterior middle of the flexible component disposed 50%-70% of the distance from the nasion to the vertex of the patient. Signal electrodes can be disposed within this flexible component. For example, signal electrodes can be disposed within this flexible component such that, when the flexible component is worn by a patient as shown in FIG. 6, signal electrodes are approximately located at the F3, F4, P3, and P4 electrode locations of the 10-20 system for electrode placement. At least one additional reference electrode can be attached to the flexible component.

A computing device can be connected to the flexible component. Leads can connect the computing device with the signal electrodes and the reference electrode. In some embodiments, the computing device can be configured to acquire EEG signals using the signal electrodes (and in some aspects additionally using at least one reference electrode). The computing device can be configured to perform at least one of signal isolation, signal conditioning, digital or analog filtering, or analog to digital conversion. In some embodiments, the computing device can be configured to process the acquired EEG signals. For example, the computing device can be configured to perform at least some of the SDI analysis described above with regard to FIG. 5. Another computing device, such as mobile device, laptop, desktop, workstation, server, or cluster can be configured to perform the remainder (if any) of the SDI analysis described above with regard to FIG. 5. In some embodiments, the computing device can be configured to provide the acquired EEG signals to the other computer device, which may then perform at least some of the SDI analysis described above with regard to FIG. 5. In some embodiments, the flexible component, together with the computing device, can comprise a headset disposed on the head of the patient.

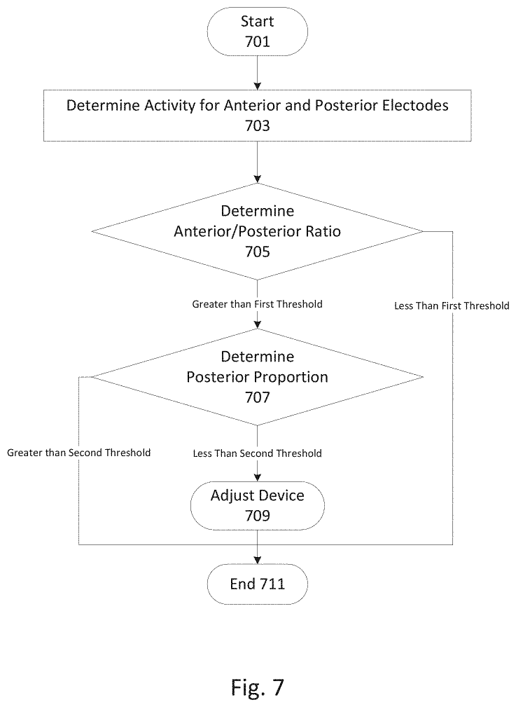

FIG. 7 depicts an exemplary method for adjusting the placement of the exemplary device of FIG. 6 on a patient. After starting at step 701, the method can include determination of activity levels for the anterior and posterior electrodes in step 703. For example, a train of stimuli can be provided to the patient. These stimuli can include auditory, visual, or tactile stimuli, as described above. Activity levels, as described above with regards to FIG. 5, can be determined for the anterior and posterior electrodes during the peri-stimulus interval for at least one stimulus in the train. For example, an activity level each stimulus in the train (or each “oddball” stimulus) can be determined.

An anterior/posterior ratio can be determined in step 705. In some embodiments, the anterior/posterior ratio can be determined using at least one stimulus in the train. For example the ratio can be determined using each stimulus in the train (or each “oddball” stimulus). The ratio can be determined from the individual ratios for multiple stimuli (e.g., by averaging these individual ratios). The ratio can be compared to a first threshold. When the ratio is less than the first threshold, the adjustment process can terminate at step 711. When the ratio is greater than the first threshold, the adjustment process can proceed to step 707. The first threshold can be between 1 and 2, or between 1.2 and 1.6.

A posterior proportion can be determined in step 707 using a number of stimuli. These stimuli can include at least one of the stimuli in the train (or at least one of the “oddball” stimuli in the train). The posterior proportion can be the portion of this number of stimuli for which the posterior activity level exceeds the anterior activity level. For example, when the stimulus train includes 10 stimuli, 8 can be used to determine the posterior proportion. When the posterior activity level exceeds the anterior activity level for 2 of these 8 stimuli, the posterior proportion can be 0.25. The posterior proportion can be compared to a second threshold. When the posterior proportion is less than the second threshold, the adjustment process can terminate at step 711. When the posterior proportion is greater than the second threshold, the adjustment process can proceed to step 709. The second threshold can be between 0.1 and 0.9, or between 0.3 and 0.5.

The positioning of the device on the patient can be adjusted in step 709. In some embodiments, the flexible component should be adjusted to lower the position of the posterior electrodes on the patient. After step 709, the adjustment process can terminate are step 711. Should the exemplary device require further adjustment, the method depicted in FIG. 7 can be repeated.

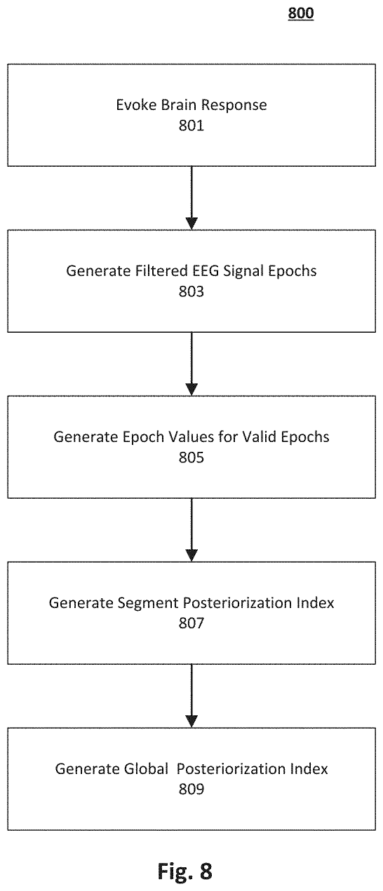

FIG. 8 depicts method 800 of detecting systemic brain dysfunction. Method 800 can be based on a comparison of brain activity between differing regions of the brain. For example, method 800 can involve comparing anterior brain activity levels with posterior brain activity levels. Method 800 can involve the steps of evoking a brain response during an analysis period, generating filtered EEG signal segments and/or epochs, generating epoch values for valid epochs, generating a segment posteriorization index, and generating a global posteriorization index.

In some embodiments, the steps of method 800 can run at least partially concurrently. For example, filtered EEG signal epochs can be generated in step 803 while brain responses are being evoked in step 801; epoch values for valid epochs can be generated in step 805 while filtered EEG signal epochs are generated in step 803; segment posteriorization indices may be generated in step 807 while epoch values for valid epochs are generated in step 805; and/or global posteriorization indices may be generated in step 809, while segment posteriorization indices are generated in step 807. In this manner, the steps of method 800 can form a processing pipeline that produces segment posteriorization indices from evoked brain responses. Alternatively, one or more steps of method 800 can be performed upon completion of a prior step of method 800.

After starting, method 800 can proceed to step 801. In step 801, a brain response can be evoked in a patient during an analysis period. The brain response can be evoked using an auditory, visual, or tactile oddball task, as described above with regards to FIG. 5, or another task capable of evoking an ERP in a patient. While described as an initial step, this process of evoking a brain response can continue throughout the analysis period.

In step 803, a recording system can be configured to generate filtered EEG signal segments and/or epochs. The recording system can include electrodes, which can be disposed within a headset. For example, the electrodes can be disposed within the exemplary device for acquiring EEG signals of FIG. 6, or a like device. In some aspects, the recording system can be configured to measure the EEG signals, filter the EEG signals, and divide the filtered EEG signals into segments and epochs.

Measuring the EEG signal can include receiving EEG signals from electrodes attached to the patient. In some implementations, there can be four electrodes. The electrodes can be attached to the patient at standard locations. The electrodes can be attached symmetrically on either side of the midline of the patient. For example, the electrodes can include two anterior electrodes. The anterior electrodes can be placed at locations corresponding to F3 and F4 of the 10-20 EEG electrode placement. As an additional example, the electrodes can include two posterior electrodes. The posterior electrodes can be placed at locations corresponding to O1 and O2 of the 10-20 EEG electrode placement. In some aspects, additional electrodes can be used, or the anterior and/or posterior electrodes can be placed at different locations. In various aspects, a single anterior electrode and/or a single posterior electrode can be used.

Filtering the EEG signal can include bandpass filtering the EEG signals received from the electrodes. This bandpass filtering can be achieved by a single filter or multiple filters. For example, the recording system can include separate lowpass and highpass filters that together bandpass filter the received EEG signals. In some aspects, the EEG signals can be filtered using a filter having a lower cutoff frequency of 5-9 hertz. In various aspects, the EEG signal can be filtered using a filter having a higher cutoff frequency of 11-15 Hz. In some aspects, the single filter or multiple filters can be configured to select the alpha EEG frequency range.

Division of the analysis period into segments and/or epochs can be performed using the recording system or another computing system. In some embodiments, the division can be performed in real-time (e.g., as the EEG signal is received from the patient). In some implementations, the analysis of the EEG signal may not be event-related. For example, the division of the analysis period into epochs may not be synchronized to the ERPs. In this manner, the analysis described with regards to FIG. 8 may differ from the analysis described with regards to FIG. 5 and FIG. 7. As a non-limiting example, the analysis of the EEG signal may be task-related. In some implementations, the division of the analysis period into epochs may depend on when the analysis started and the length of the epochs. For example, the recording system can be configured to divide the EEG signals into equal or approximately equal segments. The segments in turn can be divided into epochs. In some aspects, the epochs can be approximately 500 ms to 3 seconds long. In various embodiments, a segment can include between 5 and 20 consecutive epochs. Alternatively, the analysis period may be divided into epochs synchronized with a train of stimuli such that each epoch includes the same or a similar number of stimuli (e.g., consecutive epochs may differ in the number stimuli by less than three stimuli, or less than 10% of the number of stimuli).

In step 805, the recording system can generate epoch values for valid epochs. This step can include identification of invalid epochs, identification of invalid segments, and generation of epoch values, including anterior and posterior values. The recording device can identify invalid epochs (e.g., epochs including noisy signals, artifacts, measurement errors, confounding EMG signals, or the like) for each electrode using the filtered EEG signal from the electrode. In some aspects, identification can depend on amplitude criteria and the amplitude of the filtered EEG signal. The amplitude criteria can be absolute or relative criteria. For example, the recording device can identify a filtered EEG signal from an electrode as invalid when the filtered EEG exceeds a predetermined amplitude or fails to exceed a predetermined amplitude. As an additional example, the recording device can identify a filtered EEG signal from an electrode as invalid when the filtered EEG exceeds, or fails to exceed, a relative threshold dependent on a value of the filtered EEG signal within the epoch. This value can be the mean amplitude of the filtered EEG signal within the epoch. The relative threshold can be a multiple of the value. For example, the relative threshold can be a multiple of the mean amplitude of the filtered EEG signal within the epoch. In some aspects, identification can depend on variability criteria. For example, the recording device can identify a filtered EEG signal from an electrode as invalid when the variance (or the coefficient of variation) of the amplitude of the EEG signal within the epoch exceeds a predetermined threshold.

The recording device can also be configured to process the segments in step 805. In some embodiments, the recording device can be configured to, for each segment, exclude epochs from subsequent analysis as invalid when any of the filtered EEG signals for the epoch are invalid. In various embodiments, the recording device can be configured to exclude the segment from subsequent analysis when a certain percentage of epochs are invalid. In some implementations, this percentage can be between 40% and 60%. The recording device can be configured to process the remaining segments using the remaining epochs.

The recording device can be configured to generate anterior and posterior values for the epochs in step 805. In some embodiments, the recording device can be configured to calculate an electrode value for each electrode (e.g., electrodes placed at F3, F4, O1, and O2 of the 10-20 standard electrode placement). In some aspects, the electrode value for an electrode in an epoch can be a function of the amplitude of the filtered EEG signal during the epoch for that electrode. For example, the electrode value can be the power of the signal for that electrode for that epoch. In various embodiments, the recording device can be configured to calculate anterior and posterior values based on the electrode values for the epoch. The recording device can be configured to avoid differences in electrode values between hemispheres (e.g., laterality effects) by making the anterior and/or posterior values functions of the electrode values. For example, the anterior value can be an average of the electrode values for the anteriorly placed electrodes and/or the posterior value can be an average of the electrode values for the posteriorly placed electrodes. As an additional example, the anterior value can be the maximum (or minimum) value of the electrode values for the anteriorly placed electrodes and/or the posterior value can be the maximum (or minimum) value of the electrode values for the posteriorly placed electrodes.

In step 807, the recording system can generate segment posteriorization indices. In some embodiments, for a valid segment, the recording device can be configured to determine a count of valid epochs in the segment satisfying a relative epoch value criterion. In some implementations, counting valid epochs satisfying the relative epoch value criterion may be less noisy than functions of averaged posterior epoch values and averaged anterior epoch value. In some aspects, the relative epoch value criteria can be whether the posterior epoch value (the posterior alpha measurement) is greater than the anterior epoch value (the anterior alpha measurement). In various aspects, the recording system can be configured to compute a compute a posterior epoch value/anterior epoch value ratio and compare this ratio to a relative epoch value criteria (e.g., 1.0). In some embodiments, the recording system can be configured to generate the segment posteriorization indices satisfying a relative epoch value criterion as proportions of valid epochs satisfying the relative epoch value criterion based on the counts and the numbers of valid epochs in the segments.

In step 809, the recording system can generate global posteriorization indices as a function of two or more segment posteriorization indices for valid segments. For example, the recording system can generate the global posteriorization indices as a median, average or weighted average of segment posteriorization indices for valid segments. This generation can be performed over a window of segments, for example the last 3-10 segments of EEG data.

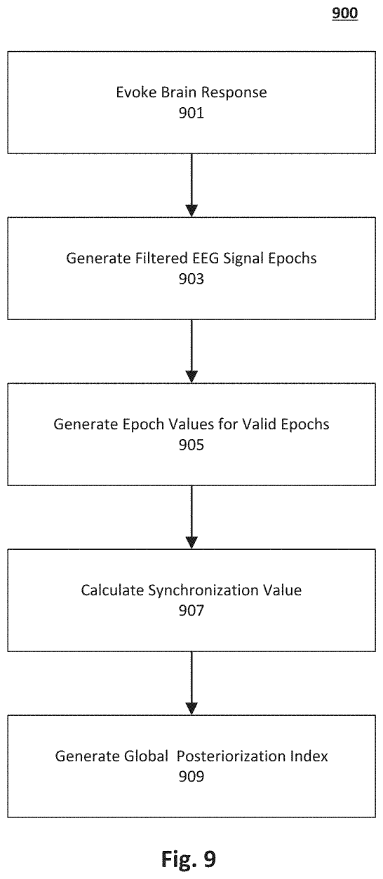

FIG. 9 depicts method 900 of detecting focal brain dysfunction. Method 900 can involve a comparison of brain activity between differing regions of the brain. For example, method 900 can involve comparing a level of synchronization between brain hemispheres. An abnormal level of synchronization can indicate an increased risk for POCD, POD, relative hypotension, relative hypoxia, or relative hypoglycemia. In some aspects, method 900 can be used for triage of concussion or stroke cases (e.g., to identify a viable post-stroke penumbra indicating a need for brain catheterization). Method 900 can involve the steps of evoking a brain response during an analysis period, generating filtered EEG signal segments and/or epochs, generating epoch values for a set of valid epochs, and calculating a synchronization value for the set of valid epochs.

Similar to the steps of method 800, described above, the steps of method 900 can form a processing pipeline that produces global posteriorization indices from evoked brain responses. Alternatively, one or more steps of method 900 can be performed upon completion of a prior step of method 900.

After starting, method 900 can proceed to step 901. In step 901, the recording system can be configured to evoke a brain response, in a manner similar to that described above with regards to step 801 of method 800.

In step 903, the recording system can be configured to generate filtered EEG signal segments and/or epochs, in a manner similar to that described above with regards to step 801 of method 800. In some embodiments, the recording system, according to method 900, can use one or more pairs of EEG electrodes placed on the patient (e.g., two paired EEG electrodes, four paired EEG electrodes, or the like). These electrodes can be symmetrically placed. In some aspects, the electrodes can be frontal electrodes (e.g., electrodes placed at locations F3 and F4 of a standard 10-20 EEG electrode placement). In some implementations, the recording system may also use a reference electrode placed on the patient. In some embodiments, the recording system, according to method 900, can filter the EEG signals from the electrodes using a lower frequency cutoff of 0.5-2 Hz and/or an upper frequency cutoff of 3-5 Hz. In various aspects, the recording system, according to method 900, can filter the EEG signals from the electrodes to select the delta EEG frequency range. The recording system can be configured to divide the analysis period into segments and/or epochs, similar to the division performed according to method 800.

In step 905, the recording system can generate epoch values for valid epochs. In a manner similar to that described above with regards to step 805 of method 800, the recoding system can be configured to identify invalid epochs. In step 905, the recording system can be configured to identify a set of consecutive valid epochs. This set can have a predetermined minimum size greater than 5 consecutive epochs and/or a predetermined maximum size less than 60 consecutive epochs.

The recording system can be configured to calculate representative values for each valid epoch in the set of consecutive valid epochs. In some aspects, the representative values can be calculated for each electrode (e.g., representative values can be calculated for each of electrodes placed at F3 and F4 of a standard 10-20 electrode placement). In some embodiments, the representative values can be functions of the amplitude of the filtered EEG signals during the epoch. For example, the representative value of a filtered EEG signal from an electrode during the epoch can be a statistical measure, such as mean, median, or mode of the amplitude of the filtered EEG signal during the epoch.

In step 907, the recording system can calculate a synchronization value for the set of valid epochs. The synchronization value can indicate a degree of synchronization between activity in hemispheres of brain of patient. In some embodiments, the synchronization value can be a Pearson's correlation coefficient, Spearman correlation coefficient, or a like correlation measure. The correlation measure can be calculated using the representative values for epochs in the set of consecutive epochs. The correlation measure can be calculated using a set of representative values for the first electrode and a set of representative values for the second electrode. Increased correlation implies greater synchronization between related ipsilateral and contralateral activities and vice versa for decreased correlation.

### Example 1: POCD Study

Initial results are available from a 2-arm 100-patient study ongoing at the Rambam Medical Center in Haifa, Israel. Arm I includes orthopedic patients undergoing Total Knee Replacement (TKR) and Total Hip Replacement (THR). These procedures are mostly performed under sedation together with regional anesthesia. Arm II includes cardiac patients undergoing procedures under general anesthesia (GA). The study design includes pre-op cognitive testing, EEG monitoring during the procedure, and post-op cognitive testing for POCD (at approximately one week, three weeks, and three months post procedure).

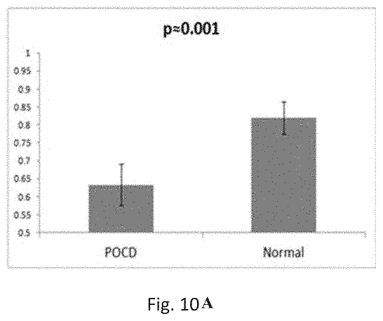

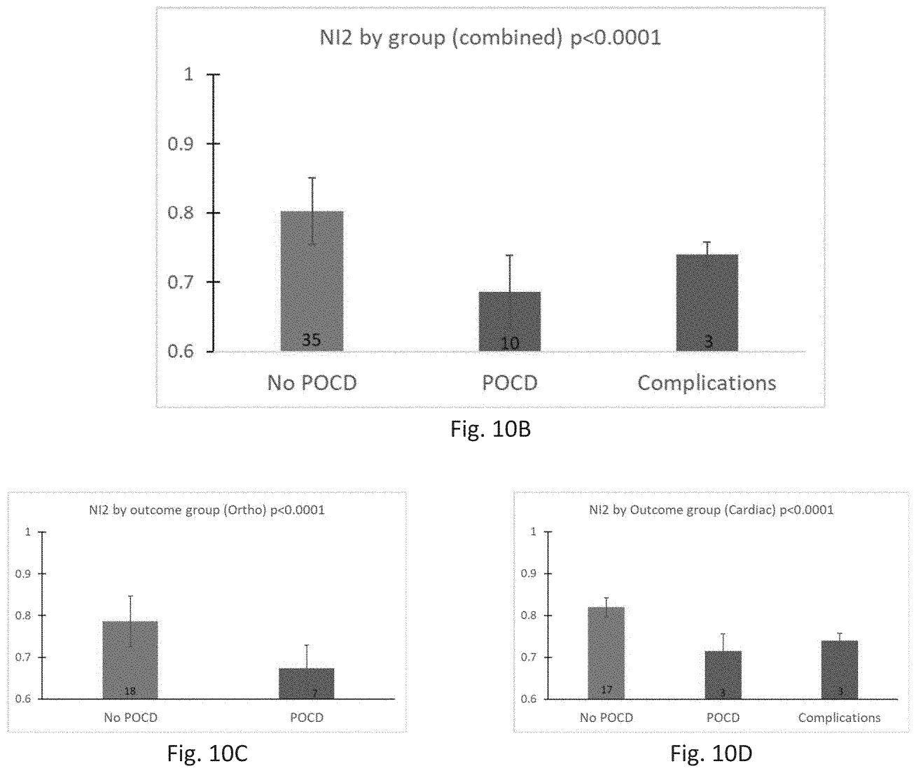

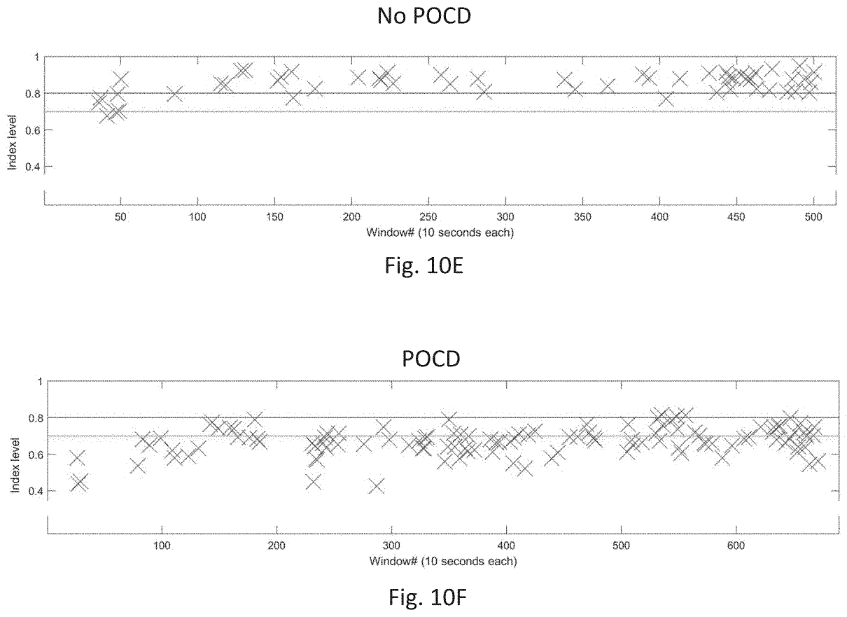

Preliminary results (N=20 and post-op cognitive evaluation after 1 week) demonstrated a statistically significant correlation between an index level calculated according to method 900 (inter-hemispheric synchronization) and POCD upon patient release, as shown in FIG. 10A. In patients who completed a three months cognitive test, follow up, it was found that approximately 50% of the patients with cognitive deterioration in the first week still exhibited post-operative cognitive deterioration after 3 months. The bars shown in FIG. 10A represent the average synchronization value during the 10 minutes when synchronization was lowest during the procedure. The POCD bar is the average of patients that were later identified with post operatively reduced cognitive function. The Normal bar is the average of patients whose cognitive function was not reduced.

Additional results (N=48 overall, including 25 orthopedic patients and 23 cardiac patients) continue to demonstrate a statistically significant correlation between an index level calculated according to method 900 (inter-hemispheric synchronization) and POCD upon patient release, as shown in FIGS. 10B to 10D. Patients that maintained high indices of inter-hemispheric synchronization (as shown in FIG. 10E) were significantly less likely (p<0.0001) to have experience complications of exhibit persistent reductions in cognitive function than patients that experienced sustained (greater than 15 min) reductions in indices of inter-hemispheric synchronization (as shown in FIG. 10F). FIGS. 10E and 10F also depict risk criterions for POCD, with index values exceeding a threshold of 0.8 indicating a lower likelihood of POCD and index values below a threshold of 0.7 indicating a higher likelihood of POCD. In some implementations, such risk criteria can depend on both an amplitude criterion (e.g., a threshold value) and a duration or proportion criterion (e.g., how low or how often indices satisfied the amplitude criterion). The inter-hemispheric synchronization values depicted in FIGS. 10E and 10F were generated using sets of consecutive epochs of 10 seconds in duration.

### Example 2: Sedation Safety Study

A 51-participant study was conducted at the Rambam Medical Center in Haifa, Israel. The study had three arms. Arm I included 26 patients undergoing surgery under sedation (18 with Midazolam and 8 with Propofol), Arm II included 12 patients undergoing surgery under general anesthesia, and Arm III included 13 awake controls. The study design included EEG monitoring during the procedure using BIS (Medtronic) as well as EPOC (Emotive) including auditory stimulation, followed by post-op testing for recall (Modified Brice Questionnaire).

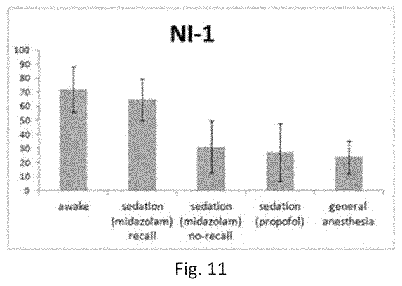

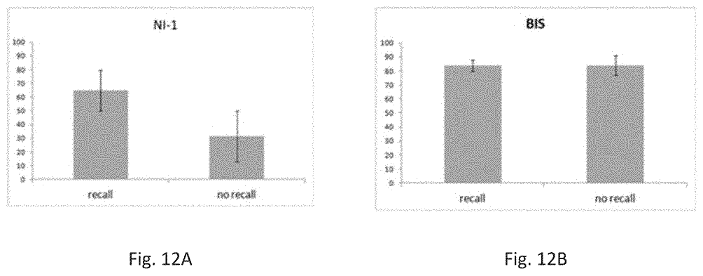

Awareness with recall was evaluated post-procedure and compared with global posteriorization index values calculated according to method 800, as well as with the results of the gold standard depth of anesthesia monitor (BIS, from Medtronic). FIG. 11 summarizes the global posteriorization index values for the patients in the different groups (Awake, Sedation (midazolam) recall, Sedation (midazolam) no-recall, Sedation propofol and General anesthesia). A Tukey post-hoc test revealed that the average global posteriorization index value in awake patients and sedated patients (midazolam) with recall was higher than in patients sedated with midazolam with no-recall, patient sedated with propofol and patient under general anesthesia (p<0.01).

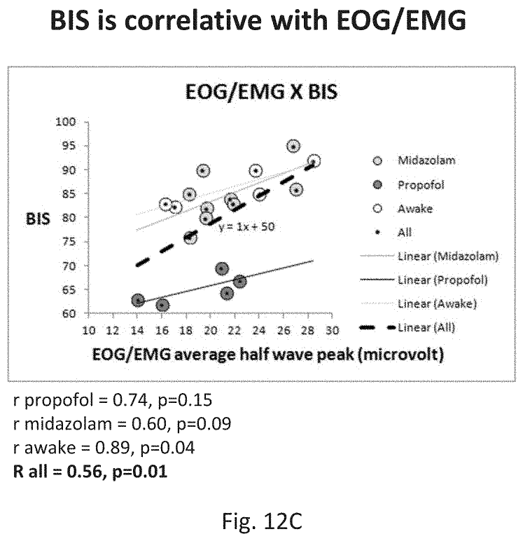

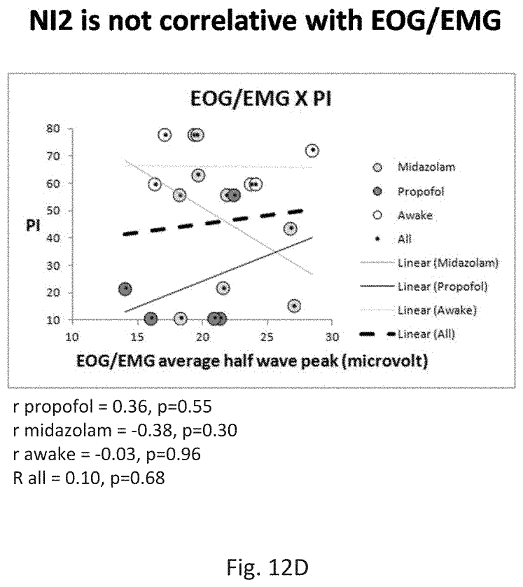

Patients sedated with Midazolam that experienced recall could be distinguished from patients sedated with Midazolam that did not experience recall based on posteriorization index values but not BIS index. A detailed analysis of the results showed that the cause of BIS' failure to identify awareness with recall was EOG/EMG noise. As shown in FIG. 12C, increasing EOG/EMG activity was correlated with increasing values of the BIS index, impairing the use of the BIS index to determine depth of anesthesia. However, as shown in FIG. 12D, such noise does not affect the index calculated according to method 800, as this index does not expressly depend on EMG.

FIGS. 12A and 12B summarize the correlation in Arm I (sedation with Midazolam, 18 patients) between the intra-operative monitoring results and the post-procedure awareness with recall. The bars in FIGS. 12A and 12B represent the average global posteriorization index values (FIG. 12A, P<0.001) and of BIS (FIG. 12B, P=0.48). Patients identified post-procedure either with recall (recall bar) or without recall (no recall bar). These results demonstrate that posteriorization index values can be used as a gauge of cognitive safety under anesthesia. This index can enhance the safety of intra-procedure sedation, where the goal is to administer the lowest dosage possible.

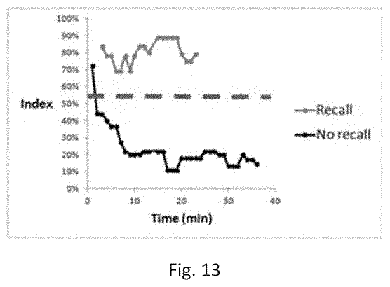

FIG. 13 shows how the physician can use posteriorization index values to provide optimal dosing for the patient by assessing both the upper dosing level to avoid delirium and the lower dosing level to avoid recall. The graphs show posteriorization index values during the procedure in two different patients. One patient (gray line) had recall of the procedure and the other patient (black line) did not have recall. The red dotted line is a threshold that in this example indicates a maximal posteriorization index value associated with no-recall. If the posteriorization index value is below the threshold line, in this example, there is no need for additional sedation dosage.

### Example 3: Stroke Catheterization Study

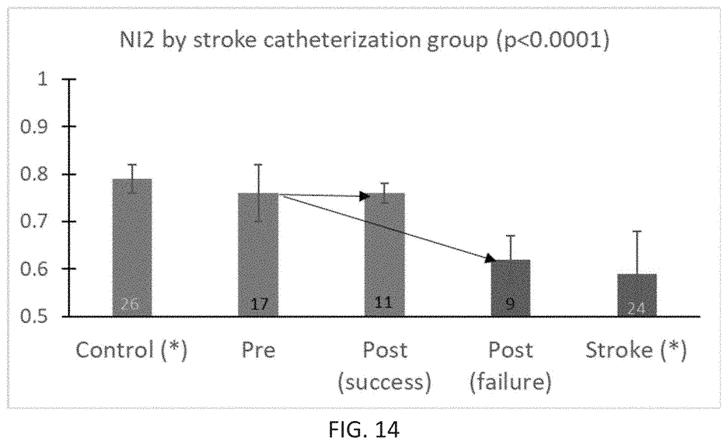

A study of 23 patients (17 patients with valid pre-procedure samples, 20 patients with valid post-procedure samples) has demonstrate detection of stroke dynamics under sedation using an index level calculated according to method 900 (inter-hemispheric synchronization). Index values were calculated for anesthetized patients for five minutes before and after performance of a thrombectomy for acute stroke. This thrombectomy was subsequently classified as successful or unsuccessful based on the results of a follow-up patient assessment performed using the NIH stroke scale. As shown in FIG. 14, index level were high prior to the thrombectomy and comparable to control values obtained in a prior study, as tissue with the penumbra remained viable. After successfully interventions, index values remained high and comparable to control and pre-intervention values. After unsuccessful interventions, index values were significantly reduced (p<0.0001) and comparable to index values for stroke patients obtained in a prior study. As recognized and appreciated by the inventor, the disclosed systems and methods can detect an unsuccessful thrombectomy post-operatively in an anesthetized patient. As further recognized and appreciated by the inventor, the disclosed systems and methods can detect stroke in an anesthetized patient.

### Example 4: Concussion Study

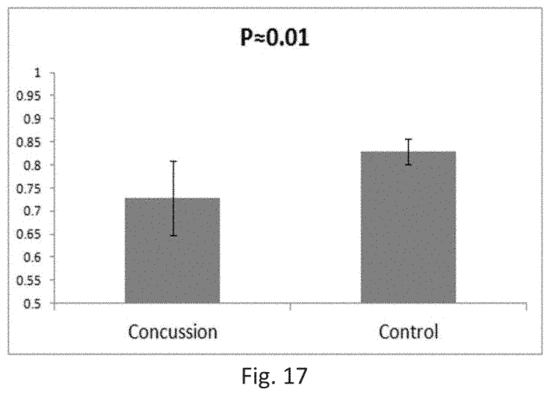

A study of 15 patients with either concussion (n=9) or isolated limb injury (n=6, serving as a control group) demonstrated that a synchronization index calculated as described above with regard to FIG. 9 can be used to identify concussion. As shown in FIG. 17, synchronization index values for patients with concussions were significantly lower than synchronization index values for patients with isolated limb injuries (p˜0.01). As recognized and appreciated by the inventor, the synchronization index may be used to diagnose concussion and/or traumatic brain injury.

Exemplary Treatment Methods:

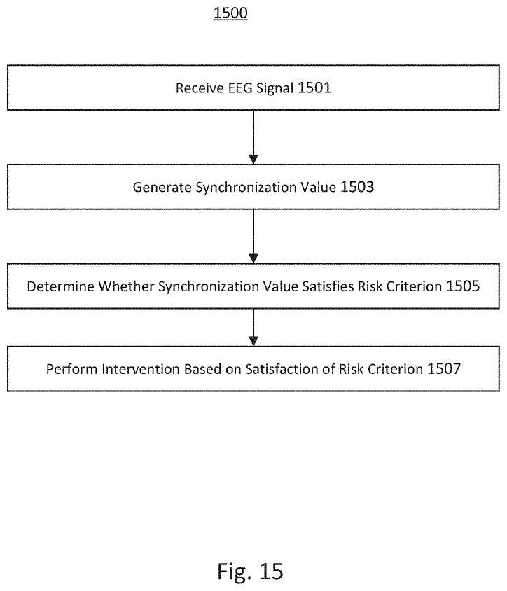

FIG. 15 depicts a method 1500 for treating an anesthetized patient, consistent with disclosed embodiments. Method 1500 can be performed during performance of a surgical procedure. For example, method 1500 can be performed while a patient is undergoing a cardiac procedure, orthopedic procedure, or another medical procedure.

In step 1501, a computing device can receive an EEG signal. The EEG signal can be received from one or more pairs of EEG electrodes placed on the patient. The computing device can be operatively connected to the EEG electrodes through a physical connection (e.g., wires) and/or wirelessly. The EEG signal can be received directly from the EEG electrodes, or indirectly through preamplifiers or other signal conditioning devices.

In step 1503, the computing device can generate a synchronization value. The synchronization value can represent a degree of synchronization between the right and left hemispheres of the patient's brain. The synchronization value can depend on anterior brain activity. The synchronization value can be calculated according to method 900, described above with regards to FIG. 9. The computing device can be configured to provide an indication of the synchronization value (e.g., at least one of a visual indication or an auditory indication).

In step 1505, a person (e.g., a practitioner such as a doctor or nurse) can determine whether the synchronization value satisfies one or more risk criteria for an anesthetic complication (e.g., post-operative delirium, post-operative cognitive deterioration, relative hypotension, relative hypoxia, or relative hypoglycemia) or intraoperative stroke. In some implementations, the person can determine whether the synchronization value satisfies the one or more risk criteria based on the synchronization value (or a time history of the synchronization value for the patient). In some embodiments, this determination can additionally depend on other patient parameters (e.g., blood pressure, blood oxygenation, or the like). In various implementations, the computing device can be configured with one or more risk criteria for an anesthetic complication or intraoperative stroke. The computing device can be configured to provide an indication (e.g., at least one of a visual indication or an auditory indication) when the synchronization value satisfies at least one of the one or more risk criteria.

In some embodiments, risk criteria can be expressed as predetermined index value thresholds. For example, a predetermined index value threshold can indicate a risk of POCD (or post-operative delirium, post-operative cognitive deterioration, relative hypotension, relative hypoxia, or relative hypoglycemia). In some implementations, this threshold value can be between 0.65 and 0.75. In various embodiments, a predetermined index value threshold can indicate a risk of intraoperative stroke. In some implementations, this threshold value can be between 0.55 and 0.65. In some embodiments, the risk criteria can depend on the change, or rate of change, in the index value. For example, a greater drop, or a more rapid drop, may indicate a greater risk of intraoperative stroke.

In step 1505, an intervention on the patient can be performed based on satisfaction of the one or more risk criteria. The intervention can be performed by the person, or another person (e.g., another practitioner). For example, when satisfaction of a risk criterion indicates a risk of POCD (or post-operative delirium, post-operative cognitive deterioration, relative hypotension, relative hypoxia, or relative hypoglycemia), a depth of anesthesia of the anesthetized patient can be reduced. As a non-limiting example, this reduction can be accomplished by delaying administration of an anesthetic dose, reducing a rate of administration of an anesthetic, or administering a reversal agent. As an additional example, when satisfaction of a risk criterion indicates a risk of intraoperative stroke, an intervention can be performed to confirm the presence of an intraoperative stroke (e.g., using medical imaging), resolve any intraoperative stroke (e.g., by performing a thombectory), and/or mitigate the effects of any intraoperative stroke (e.g., by administering protective agents or blood thinners). As would be appreciated by one of skill in the art, this list of interventions is not intended to be exhaustive or limiting.

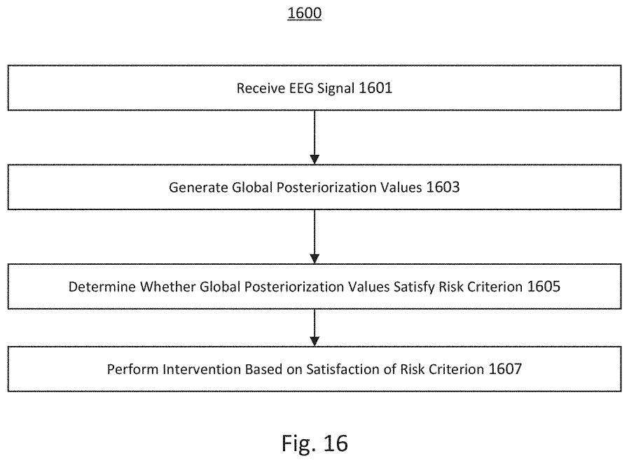

FIG. 16 depicts a method 1600 for treating an anesthetized patient, consistent with disclosed embodiments. Method 1600 can be performed during performance of a surgical procedure. For example, method 1600 can be performed while a patient is undergoing a cardiac procedure, orthopedic procedure, or another medical procedure.

In step 1601, a computing device can receive an EEG signal. The EEG signal can be received from one or more pairs of EEG electrodes placed on the patient. The computing device can be operatively connected to the EEG electrodes through a physical connection (e.g., wires) and/or wirelessly. The EEG signal can be received directly from the EEG electrodes, or indirectly through preamplifiers or other signal conditioning devices.

In step 1603, the computing device can generate a global posteriorization index value. The global posteriorization index value can depend on a relative degree of anterior brain activity and posterior brain activity. The synchronization value can be calculated according to method 800, described above with regards to FIG. 8. The computing device can be configured to provide an indication of the synchronization value (e.g., at least one of a visual indication or an auditory indication).

In step 1605, a person (e.g., a practitioner such as a doctor or nurse) can determine whether the global posteriorization index value satisfies a risk criterion for awareness during anesthesia. In some implementations, the person can determine whether the global posteriorization index value satisfies the one or more risk criteria based on the global posteriorization index value (or a time history of the global posteriorization index value for the patient). In some embodiments, this determination can additionally depend on other patient parameters (e.g., blood pressure, blood oxygenation, or the like). In various implementations, the computing device can be configured with a risk criterion for awareness during anesthesia. The computing device can be configured to provide an indication (e.g., at least one of a visual indication or an auditory indication) when the global posteriorization index value satisfies the risk criterion.

In some embodiments, the risk criterion can be expressed as a predetermined index value threshold. For example, a predetermined index value threshold can indicate a risk of awareness during anesthesia. In some implementations, this threshold value can be between 0.5 and 0.8 (e.g., the threshold value may be 0.7) In some embodiments, the risk criteria can depend on the change, or rate of change, in the index value. For example, a greater rise, or a more rapid rise, may indicate a greater risk of awareness during anesthesia.

In step 1605, an intervention on the patient can be performed based on satisfaction of the one or more risk criteria. The intervention can be performed by the person, or another person (e.g., another practitioner). For example, when satisfaction of a risk criterion indicates a risk of awareness during anesthesia, a depth of anesthesia of the anesthetized patient can be increased. As a non-limiting example, this increase can be accomplished by administering an anesthetic, increasing a rate of administration of an anesthetic, or administering an anesthetic agonist.

Other embodiments will be apparent to those skilled in the art from consideration of the specification and practice of the disclosed embodiments disclosed herein. It is intended that the specification and examples be considered as exemplary only, with a true scope and spirit of the disclosed embodiments being indicated by the following claims. Furthermore, although aspects of the disclosed embodiments are described as being associated with data stored in memory and other tangible computer-readable storage mediums, one skilled in the art will appreciate that these aspects can also be stored on and executed from many types of tangible computer-readable media, such as secondary storage devices, like hard disks, floppy disks, or CD-ROM, or other forms of RAM or ROM. Accordingly, the disclosed embodiments are not limited to the above-described examples, but instead are defined by the appended claims in light of their full scope of equivalents.

Moreover, while illustrative embodiments have been described herein, the scope includes any and all embodiments having equivalent elements, modifications, omissions, combinations (e.g., of aspects across various embodiments), adaptations or alterations based on the present disclosure. The elements in the claims are to be interpreted broadly based on the language employed in the claims and not limited to examples described in the present specification or during the prosecution of the application, which examples are to be construed as non-exclusive. Further, the steps of the disclosed methods can be modified in any manner, including by reordering steps or inserting or deleting steps. It is intended, therefore, that the specification and examples be considered as example only, with a true scope and spirit being indicated by the following claims and their full scope of equivalents.

1-40. (canceled)

41. A method for generating a brain dysfunction index, comprising:
placing at least two electrodes on a head of a subject, each above a different anatomical region of the brain;
recording at least one first electrical signal indicating activity of a first brain region and at least one second electrical signal indicating activity of a second brain region, by said at least two electrodes;
processing said at least one first electrical signal and said at least one second electrical signal;
determining a correlation between said activity of said first brain region and said activity of said second brain region using said processed at least one first electrical signal and said processed at least one second electrical signal;
generating at least one brain dysfunction index value based on said determined correlation, indicating a brain dysfunction clinical state.

42. A method according to claim 41, comprising:
presenting a stimulus configured to affect brain activity at one or both of said first brain region and said second brain region, to said subject prior to and/or during said recording, and wherein said determining a correlation comprises determining said correlation in response to said stimulus.

43. A method according to claim 42, wherein said recording comprises recording said at least one first electrical signal and said at least one second electrical signal, under anesthesia.

44. A method according to claim 41, comprising:
determining a relation between said generated at least one brain dysfunction index value and an anesthesia-related risk criteria; and
reducing or increasing anesthesia depth according to said determined relation.

45. A method according to claim 43, wherein said recording comprises recording said at least one first electrical signal from at least one electrode positioned above an anterior brain region, and said at least one second electrical signal from at least one electrode positioned above a posterior brain region, and wherein said generated at least one brain dysfunction index value comprises at least one posteriorization index value reflecting a ratio between posterior brain activity and frontal brain activity.

46. A method according to claim 45, wherein said determining comprises determining by a computing device a relation between said generated at least one posteriorization index value and a risk criterion for awareness during anesthesia.

47. A method according to claim 45, wherein said determining comprises determining by a computer device whether current anesthesia levels are sufficient to avoid recall during sedation based on said determined relation.

48. A method according to claim 45, wherein said at least one first electrical signal and said at least one second electrical signal comprise electroencephalographic (EEG) signals, and wherein said processing comprises filtering said EEG signals to select EEG signals in alpha EEG frequencies in a range of 5-15 Hz.

49. A method according to claim 44, wherein said recording comprises recording said at least one first electrical signal from at least one electrode positioned above a brain region of the left hemisphere, and said at least one second electrical signal from at least one electrode positioned above a brain region of the right hemisphere, and wherein said generated at least one brain dysfunction index value comprises a focal brain dysfunction index value, reflecting a level of synchronization between brain hemispheres.

50. A method according to claim 49, wherein said determining a correlation value comprises determining a synchronization value between a processed electrical signal recorded from said left hemisphere, and a processed electrical signal recorded from said right hemisphere.

51. A method according to claim 49, wherein said determining comprises determining a relation between said generated at least one focal brain dysfunction index value and a risk criteria for an anesthetic complication.

52. A method according to claim 51, wherein said anesthetic complication comprises at least one of post-operative delirium, post-operative cognitive deterioration, relative hypotension, relative hypoxia, and relative hypoglycemia.

53. A method according to claim 51, wherein said reducing or increasing anesthesia depth comprises at least one of delaying administration of an anesthetic dose, reducing a rate of administration of an anesthetic, and administering a reversal agent, according to said determined relation.

54. A method according to claim 49, wherein said determining a relation comprises determining a relation between said generated at least one focal brain dysfunction index value and a risk criteria of intraoperative stroke.

55. A method according to claim 54, wherein said reducing or increasing anesthesia depth comprises at least one of performing an intervention to confirm a presence of an intraoperative stroke, performing a thrombectomy, and administering protective agents or blood thinners.

56. A method according to claim 49, wherein said at least one first electrical signal and said at least one second electrical signal comprise electroencephalographic (EEG) signals, and wherein said processing comprises filtering said EEG signals to select EEG signals in delta EEG frequencies having frequency values smaller than 4 Hz.

57. A method according to claim 41, wherein said processing comprises:
generating filtered electrical signal epochs from said first electrical signal and said second electrical signal;
generating epoch values from said filtered electrical signal epochs of said first electrical signal and said second electrical signal; and
wherein said determining a correlation comprises determining a correlation between epoch values of said first electrical signal and epoch values of said second electrical signal.

58. A method according to claim 41, wherein said at least one first electrical signal and said at least one second electrical signal indicate event-related potentials (ERPs).

59. A device for generating a brain dysfunction index, comprising:
at least one computer readable medium storing instructions for operations of at least one processor;
the at least one processor, configured to:
receive electrical signals from two or more electrodes located above two or more different brain regions of a patient brain during presentation of a stimuli to the patient, wherein each of said electrical signals indicate electrical activity of a different brain region of said patient;
generate filtered signal epochs from each of the received electrical signals;
generate epoch values using the filtered signal epochs;
generate brain dysfunction index values by calculating a correlation between epoch values generated from an electrical signal recorded from a first brain region and epoch values generated from an electrical signal recorded from a second brain region; and
display a clinical state indication based on said generated brain dysfunction index.

60. A device according to claim 59, wherein said at least one processor is configured to receive a first electrical signal recorded from an anterior brain region, and a second electrical signal recorded from a posterior brain region;
generate posteriorization index values by calculating a correlation between epoch values of said first electrical signal and epoch values of said second electrical signal, wherein said posteriorization index values reflect a ratio between posterior brain activity and frontal brain activity; and
display a depth of anesthesia indication based on said generated posteriorization index values.

61. A device according to claim 60, wherein said received first electrical signal and said second electrical signal are EEG signals, and wherein said at least one processor is configured to filter said received EEG signals to select EEG signals in alpha EEG frequencies in a range of 5-15 Hz.

62. A device according to claim 59, wherein said at least one processor is configured to receive a first electrical signal recorded from a first brain hemisphere, and a second electrical signal recorded from a second brain hemisphere;
generate synchronization index values by calculating a correlation between epoch values of said first electrical signal and epoch values of said second electrical signal, wherein said synchronization index values reflect a level of synchronization between brain hemispheres; and
display a depth of anesthesia indication and/or an anesthesia complication indication based on said generated synchronization index values.

63. A device according to claim 62, wherein said received first electrical signal and said second electrical signal are EEG signals, and wherein said at least one processor is configured to filter said received EEG signals to select filtered EEG signals in a delta EEG frequency values having frequency values smaller than 4 Hz.
